(19) United States
(12) Patent Application Publication
(10) Pub. No.: US 2005/0222638 A1
(43) Pub. Date: Oct. 6, 2005

(54) Sensor based gastrointestinal electrical stimulation for the treatment of obesity or motility disorders

(76) Inventors: Steve Foley, Kerrville, TX (US); Jiande Chen, Houston, TX (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/091,287

(22) Filed: Mar. 28, 2005

Related U.S. Application Data
(60) Provisional application No. 60/557,736, filed on Mar. 30, 2004.

(51) Int. Cl. A61N 1/18

(52) U.S. Cl. 607/40

(57) ABSTRACT

A method for treatment of obesity, especially morbid obesity, gastroparesis and other syndromes related to motor disorders of the stomach. The method of this invention utilizes a sensor to detect food entering the patient's stomach, thereby the sensor communicates with and activates at least one electrical stimulation device attached to either the stomach or the small intestine.

### CROSS-REFERENCE TO RELATED APPLICATION

This application claims the benefit of U.S. Provisional application Ser. No. 60/557,736, filed Mar. 30, 2004, which is incorporated by reference in its entirety herein.

### FIELD OF THE INVENTION

A method for treatment of obesity, especially morbid obesity, gastroparesis, and other syndromes related to motor disorders of the stomach is provided. The methods of this invention utilize a sensor to detect food entering the patient's stomach, the sensor then communicates with, and activates, at least one electrical stimulation device attached to the stomach and/or the small intestine.

### BACKGROUND OF THE INVENTION

Patients having an excessively high amount of body fat or adipose tissue in relation to lean body mass are considered obese. Such obese patients generally have a body mass index (BMI, which is the ratio of weight in kilograms to the square of the height in meters) of 30 or more. Morbidly obese patients generally have a BMI of greater than 40. Modern surgical procedures for treatment of obesity generally entail the reduction of gastric compliance, with the aim of limiting the subject's ability to ingest food, or reduction of the food absorption surface by shortening or bypassing part of the digestive canal. Since the major surgical procedures (e.g., removal or blocking off of a portion of the stomach) currently in use have some immediate and/or delayed risks, surgery is considered an extreme solution for use only when less invasive procedures fail. Furthermore, even surgical treatment fails in some cases, thereby requiring the surgeon to attempt to correct the problem or restore the original anatomical situation.

The digestive disease gastroparesis is characterized by delayed gastric emptying in which the stomach takes too long to empty its contents. This typically occurs when nerves of the stomach are damaged or otherwise functionally impaired, thereby causing the movement of food through the stomach to be significantly slowed or stopped. Treatments for gastroparesis typically include oral medications, changes in diet or, for severe cases, feeding tubes and intravenous feeding. Because the current treatment methods for gastroparesis are largely dependant on the patients own management of diet and medication, new solutions are needed to provide more consistent and reliable treatment to patients.

To investigate the problems of obesity, gastroparesis, and other motility disorders, researchers are experimenting with the pacing of the stomach and other portions of the gastrointestinal (GI) tract via electrical pulses. The two types of gastrointestinal electrical activity include slow waves and spikes. Gastrointestinal electrograms indicate an intrinsic gastric muscular electrical activity of the stomach as about 2 to about 4 slow waves per minute whereas the small intestine has an activity of about 10 to about 13 slow waves per minute. Generally spikes have a rate of about 120 to about 1200 waves per minute in both the stomach and the small intestine. The slow wave is omni-present and is the basic electrical rhythm of the stomach or small intestine. The presence of spikes are directly associated with the contraction of the stomach or small intestine. However, spikes only occur in the phase of slow waves and are superimposed with slow waves. Thus, the slow waves appear to act as a clock for contractile waves and determine the frequency and propagation direction of the contractile waves.

Recently, methods have been successfully employed whereby an electrical stimulation device is implanted on, or adjacent to, the stomach wall and/or small intestine. For example, U.S. Pat. No. 5,423,872 (Jun. 13, 1995) provides a process for the treatment of obesity and related disorder employing an electrical stimulator or pacemaker attached to the antrum or greater curvature of the stomach. U.S. Pat. No. 6,615,084 (Sep. 2, 2003) provides a process for the treatment of obesity and related disorder employing an electrical stimulator or pacemaker attached to the lesser curvature of the stomach. U.S. Pat. No. 5,690,691 (Nov. 25, 1997) provides a portable or implantable gastric pacemaker including multiple electrodes positionable on the inner or outer surface of an organ in the gastrointestinal tract which are individually programmed to deliver a phased electrical stimulation to pace peristaltic movement of material through the gastrointestinal tract. U.S. Pat. No. 6,606,523 (Aug. 12, 2003) provides an apparatus for stimulating neuromuscular tissue of the gastrointestinal tract and methods for installing the apparatus to the surface of the neuromuscular tissue. More recently, U.S. patent application Ser. No. 10/627,908 (filed Jul. 25, 2003) provides methods whereby an electrical stimulation device is implanted on the small intestines or lower bowel. All of these patents and patent applications, as well as all patents, patent applications, and publication cited herein, are hereby incorporated by reference in their entireties.

Existing electrical stimulation technologies attempt to evoke contractions by applying a series of electrical pulses in an “all or none” approach. This approach employs full electrical stimulation during preset periods of time with no stimulation during the remaining time periods. Currently, the major procedures used to apply such electrical stimulation include: (1) creating surrogate slow waves and (2) timing electrical stimulation during the window of susceptibility.

The surrogate slow wave approach applies electrical stimulation energy that is significantly stronger than the normal slow wave. The large amounts of energy applied with surrogate slow waves may cause significant tissue damage from the chronic application at the electrode sites. Moreover, because of the large amounts of energy to create the surrogate slow waves, the device may not have a realistic lifetime due to high current drains.

The second approach (i.e., applying electrical stimulation during the window of susceptibility time period of the slow wave) still uses the “all or none” approach. Pulses that may occur outside the window of susceptibility are effectively wasted. Moreover, electrical stimulation applied outside the window of susceptibility may actually interfere with the efficiency of the intrinsic GI activity by depolarizing the tissue before an intrinsic contraction is initiated. Thus, this approach may result in the expenditure of needless energy and/or actually adversely affect the overall process by generating stimulation pulse trains that are not synchronized with the slow wave window of susceptibility.

In the treatment of obesity, electrical stimulation of the stomach delays the stomach transit by continuous disruption of the intrinsic electrical activity during periods of therapy. Such continuous disruption may result in weight loss by decreasing the cross sectional area of the stomach by inducing contractions, lessening the capacity of the stomach during periods of therapy, changing the intrinsic direction and frequency of the peristalsis during periods of therapy, and/or modulating the sympathetic nervous system. Also in the treatment of obesity, electrical stimulation of the small intestine deceases the small intestinal transit time (i.e., speeds passage through the small intestines) by efficient electrical induction of peristalsis thereby reducing the level of absorbed components.

In treatment of gastroparesis and other motility disorders, electrical stimulation improves gastric emptying by accelerating the transit time of food moving through the GI tract and/or relieving neurally mediated symptoms associated with gastroparesis. Thus, electrical stimulation increases the frequency or amplitude of peristaltic contractions thereby intensifying the rapidity, or force used to propel, ingested components through the GI tract.

Prior art treatment methods require fairly extensive gastro-electrophysiological training and/or experience by a physician to achieve optimal electrical stimulation for individual patient therapies. Widespread usage of such treatment methods would require either an increase in the knowledge base of the gastric physician or a method and/or devices incorporating features to automatically determine the optimum stimulation parameters for individual patient therapies.

Thus, there exists a need for devices and methods wherein partial electrical stimulation can be applied. There also exists a need for devices and methods that can be used to determine whether such electrical stimulation is being properly applied for an effective treatment. It would be desirable for devices and methods (preferably in an automatic manner) which can aid the physician in determining more efficient operational parameters for an individual patient over time. It would be desirable to improve the efficacy of obesity therapy by utilizing information concerning peristaltic contractions and other patient conditions to control and improve treatment parameters. Such information may be used, for example, to modify gastrointestinal motility, decrease the effective capacity of the stomach, and/or decrease the transit time of food through the stomach and/or small intestine. The present invention provides such devices and methods.

### SUMMARY OF THE INVENTION

The present invention provides improved devices and methods for treatment of obesity, especially morbid obesity, gastroparesis, and other syndromes related to motor disorders of the stomach. The devices and methods of this invention utilize a sensor to detect food entering the patient's stomach. The sensor then communicates with, and activates as appropriate, at least one gastric electrical stimulation device (GESD) attached to or adjacent to the stomach and/or an intestinal electrical stimulation device (IESD) attached to or adjacent to the small intestine. More than one GESD and/or IESD can be used if desired. The sensor may communicate directly with both the at least one GESD and the at least one IESD. The sensor may also communicate directly with the at least one GESD, which in turn is in communication with the at least one IESD. Preferably, the at least one GESD is attached to or is adjacent to, the lesser curvature (i.e., towards the pylorus) and the at least one IESD attached to or is adjacent to the duodenum and/or jejunum.

The present invention provides a method for treatment of a motor disorder in a patient, said method comprising: implanting a first electrical stimulation device comprising one or more first electrical stimulation leads and a first electrical connector for attachment to a first pulse generator such that the one or more first electrical stimulation leads are attached to, or adjacent to, the patient's stomach, whereby electrical stimulation can be provided to the stomach through the one or more first electrical stimulation leads; implanting a second electrical stimulation device comprising one or more second electrical stimulation leads and a second electrical connector for attachment to a second pulse generator such that the one or more second electrical stimulation leads are attached to, or adjacent to, the patient's small intestines, whereby electrical stimulation can be provided to the patient's small intestines through the one or more second electrical stimulation leads; placing a sensor on or adjacent to the patient's stomach, wherein the sensor can be activated when food enters the patient's stomach, and wherein the sensor, when activated, activates the first pulse generator to supply electrical stimulation to the patient's stomach for a first predetermined period of time and wherein the sensor, when activated, activates the second pulse generator to supply electrical stimulation to the patient's small intestines, after a predetermined delay period, for a second predetermined period of time; supplying electrical stimulation to the patient's stomach through the one or more first electrical stimulation leads for the first predetermined period of time when the sensor is activated by food entering the patient's stomach; and supplying electrical stimulation to the patient's small intestines through the one or more second electrical stimulation leads after the predetermined delay period and for the second predetermined period of time sensor when the sensor is activated by food entering the patient's stomach.

The present invention also provides a method for treatment of a motor disorder in a patient, said method comprising: implanting an electrical stimulation device comprising one or more electrical stimulation leads and an electrical connector for attachment to a pulse generator such that the one or more electrical stimulation leads are attached to, or adjacent to, the patient's stomach, whereby electrical stimulation can be provided to the stomach through the one or more electrical stimulation leads; placing a sensor on or adjacent to, the patient's stomach, wherein the sensor can be activated when food enters the patient's stomach, and wherein the sensor, when activated, activates the pulse generator and supplies electrical stimulation to the patient's stomach for a predetermined period of time; and supplying electrical stimulation to the patient's stomach through the one or more first electrical stimulation leads for the predetermined period of time when the sensor is activated.

The present invention also provides a method for treatment of a motor disorder in a patient, said method comprising: implanting an electrical stimulation device comprising one or more electrical stimulation leads and an electrical connector for attachment to a pulse generator such that the one or more electrical stimulation leads are attached to, or adjacent to, the patient's small intestines, whereby electrical stimulation can be provided to the small intestines through the one or more electrical stimulation leads; placing a sensor on or adjacent the patient's small intestines, wherein the sensor can be activated when food enters the patient's stomach, and wherein the sensor, when activated, activates the pulse generator to supply electrical stimulation to the patient's small intestines, after a predetermined delay period, for a predetermined period of time; and supplying electrical stimulation to the patient's small intestines through the one or more electrical stimulation leads after the predetermined delay period and for the predetermined period of time when the sensor is activated.

This invention also provides a method for treatment of a motor disorder in a patient, said method comprising: implanting an electrical stimulation device comprising an information processor, a plurality of sensors, and an electrical stimulator; placing the information processor in communication with the plurality of sensors, wherein the plurality of sensors provides physiological parameter(s) or patient condition information to the information processor; and placing the information processor in communication with the electrical stimulator, whereby the electrical stimulator can provide electrical stimulation to the patient's stomach or small intestines through the one or more electrical stimulation leads and wherein the information processor controls the electrical stimulator.

This invention also provides a method for treatment of a motor disorder in a patient, said method comprising: implanting an electrical stimulation device comprising an information processor, a plurality of sensors, a telemeter, and an electrical stimulator; placing the information processor in communication with the plurality of sensors, wherein a plurality of sensors provide physiological parameter(s) or patient condition information to the information processor; placing the information processor in communication with the telemeter; and placing the telemeter in communication with the electrical stimulator, wherein the telemeter controls the electrical stimulator and wherein the electrical stimulator can provide electrical stimulation to the patient's stomach and/or small intestines through the one or more electrical stimulation leads.

The present devices and methods use a sensor or sensors to detect food entering the patient's stomach. The sensor then activates or initiates the application of electrostimulation immediately and/or after a predetermined time period. The sensor may be placed within or adjacent to the stomach (including placement internal or external to the body). The sensor can be used to trigger the GESD and/or IESD before, during, and/or after the consumption of food.

The devices and methods of the present invention can provide, or easily be modified to provide, a number of advantages over prior art systems. For example, the use of such sensors allows patients and their physicians an easy mechanism for terminating use of the system if the need arises since the sensor can easily be modified to be inactivated as desired, thereby preventing electrical stimulation. Thus, for example, if a patient becomes conditioned to the pattern of electrostimulation, the device could be shut off for a time period or the operational parameters varied to allow the patient to become unconditioned. Alternatively, the system could be periodically shut down (preferably at random times) to reduce the risk of such conditioning.

Since the sensor can be activated by gastrointestinal activity and/or other patient conditions, the electrical stimulation can be synchronized within the window of susceptibility thereby providing the patient with both the optimal treatment and the greatest device longevity. Since the sensor allows the electrostimulation device to be shut down when electrostimulation is not needed, battery lifetimes can be significantly extended. Thus, additional surgery to replace batteries can be eliminated or significantly reduced. Or if a rechargeable battery is used, the intervals between recharging, as well as the overall lifetime of the rechargeable battery, can be significantly extended.

The sensor can also be used to measure and record physiological parameters and other patient information or data. Such data can be transmitted (preferably using wireless techniques) to a remote unit for display or storage. Such transmission can be in real-time or upon demand (e.g., stored in memory until downloaded) and can be unidirectional or bidirectional (e.g., providing a feedback loop to modify operating parameters based on data interpretation by the physician). Such data may be provided for recording, logging biometric changes, and date and time information for later retrieval from memory. Advantageously the stored information may be used in treating patients to adjust, modify, or optimize the therapy, treating patients who are resistant to physician follow-up (i.e., physicians can review patient by remote or telephonic means), or for maintaining patient records for use at a later time. The physician can use such data to determine the optimal stimulation intervals for a single patient to achieve increased efficacy and increased device longevity.

The sensor, after electrical stimulation has occurred, may, if desired, automatically verify that the appropriate electrical stimulation occurred and the operating parameters used. Such automatic verification is advantageous as the sensor may automatically adjust the stimulation parameter selection, thereby providing a system which can be used by physicians with minimal electrophysiological experience.

The information obtained by the sensor may be retrieved through a telephone interface device, as well as an internet network or wireless connection. Such network or wireless connections provide downloading of information through a computer interface via modem or wireless transmission to a physician or monitoring service. Advantageously, stimulator devices may be programmed through the telephone or computer interface, which may automatically provide adaptive functions based on the history of successful stimulations versus delivered stimulation.

This invention also allows for electrical stimulation to be applied in a proportional manner in relation to the sensor activity or to time. As noted, prior electrical stimulation devices utilize an “all or nothing” approach to stimulation. Some patients find this degree of stimulation to be uncomfortable or intolerable. Patients may experience a increased tolerance to therapy by allowing the physician and, if desired the patient, to easily modify the periods of stimulation, limiting periods of stimulation to time periods in which simulation may be effective, and/or modifying the intensity of stimulation when applied.

Moreover, the sensor can automatically select stimulation parameters based on input supplied by the physician, whether in the form of specific instructions or an incorporated algorithm so that operating parameters can be automatically modified based on real time patient data. The sensor may also, based on such an algorithm, select, suggest, telemeter, and adjust stimulation parameters to optimize longevity and efficacy of specific patient therapy.

These and other features of the devices and methods of the present invention will be apparent from a consideration of the entire specification.

### BRIEF DESCRIPTION OF THE DRAWINGS

FIG. 1 is a sectional view of a gastrointestinal tract showing various embodiments using a sensor located on the stomach and which communicates a GESD and an IESD. Panels A-D present several, non-limiting, arrangements and communication pathways.

FIG. 2 provides several plots of the intensity of the response of the sensor detecting food intake, the impulses to the stomach and small intestines as a function of time.

FIG. 3 is a block diagram showing a further embodiment of the invention wherein a plurality of sensors in a neural network are used to communicate with the GESD and the IESD.

FIG. 4 illustrates synchronization of the stimulation with the window of susceptibility. Panel A illustrates the slow wave duration and the window of susceptibility; Panel B illustrates electrostimulation pulses which are not synchronized with the window of susceptibility; and Panel C illustrates electrostimulation which initiated using a sensor so that it is synchronized with the window of susceptibility.

### DETAILED DESCRIPTION OF THE PREFERRED EMBODIMENT

Devices and methods for treatment of obesity (especially morbid obesity), gastroparesis, and other syndromes related to motor disorders of the stomach are provided. The devices and methods of this invention utilize a sensor to detect the introduction of food into the patient's stomach and/or the occurrence of digestive processes indicating food within the patient's stomach. The sensor communicates with, and can activate, a gastric electrical stimulation device (GESD) attached to or adjacent to the stomach and/or an intestinal electrical stimulation device (IESD) attached to or adjacent to the small intestine. More than one GESD and/or IESD can be used if desired. The sensor may communicate directly with the GESD and the IESD or with the GESD, which in turn is in communication with the IESD. Preferably, the GESD is attached to or is adjacent to, the lesser curvature (i.e., towards the pylorus of the stomach) or the fundus, and the IESD is attached to or is adjacent to the duodenum and/or jejunum.

As used herein, “communication” means the transmission and/or exchange of information, messages, or signals by any form. Examples include, but not limited to, communication through wired and wireless connections (e.g., electrical stimulation leads, digital signals, telemetric devices, transtelephonic programming, other radio frequency-based approach, and the like), whereby the communication proceeds from the sensor to the electrical stimulation devices, pulse generations, and/or microprocessors.

In one embedment, communication can proceed from the sensor to both the gastric pulse generator and the intestinal pulse generator, whereby the gastric pulse generator supplies electrical stimulation to the patient's stomach for a first predetermined time and whereby the intestinal pulse generator supplies electrical stimulation to the patient's small intestine, after a predetermined delay period, for a second predetermined time. Communication can also proceed from the sensor to the gastric pulse generator and then to the intestinal pulse generator, whereby the gastric pulse generator supplies electrical stimulation to the patient's stomach for a first predetermined time and whereby the intestinal pulse generator supplies electrical stimulation to the patient's small intestine, after a predetermined delay period, for a second predetermined time. Communication can also proceed from the sensor to a single pulse generator, whereby the single pulse generator supplies electrical stimulation to both the patient's stomach for a first predetermined time and then to the patient's small intestine, after a predetermined delay period, for a second predetermined time. Communication can also proceed from the sensor to a microprocessor whereby the microprocessor communicates with the gastric pulse generator, the intestinal pulse generator and/or a single pulse generator by one of the means described herein.

As used herein, “sensor” means one or more devices that receive, send, transmit, and/or respond to signals and stimuli. Such sensor may include, but are not limited, to strain gauges to indicate or measure mechanical movement, thermistor or thermopile sensors to indicate temperature changes associated in food digestion or intake, accelerometer sensors to indicate stomach motion, piezoelectric crystal sensors to indicate acoustic vibration or noise associated with food digestion, ultrasound, rf component, or magnetic sensors to facilitate measurement of stomach distension and contraction, impedance plethymography sensors to indicate stomach tissue changes associated with stomach distension and contraction, pH sensors to measure pH changes within the stomach, chemical sensors to measure levels of digestion-related chemical and/or enzymes, and the like. Additionally, sensor activation may include, but are not limited to, activation by coordinated movement of the stomach indicating the beginning or continuation of the digestion process, activation by activity associated with the gastric pacemaker upon food entering into the patient's stomach, activation by physical entry of the sensor into the stomach, activation by mechanical or chemical digestion, activation by patient's condition, activation from outside the body, activation by a telemetry device, activation by magnet, and/or activation by digital or radio frequency means.

The sensor is placed “on, or adjacent to, the stomach”; such placement is intended to cover various locations on or near the stomach or on other organs which communicate with the stomach during digestion (e.g., nerves used to activate and/or control digestive processes (especially the vagus nerve), organs supplied digestive signals, digestive enzymes, and the like). For example, the sensor may be placed on the external surface of the stomach or adjacent to the stomach, within or external to the body, or within the stomach, and the like. The sensor should detect, either directly or indirectly, entry of food into the stomach. Thus, for example, the sensor may be located on the outside of the stomach (but within the patient's body) and be activated by coordinated movement of the stomach indicating the beginning of the digestion process, activity associated with the gastric pacemaker, and/or by any of the means described herein.

Alternatively, the sensor may be located within the stomach cavity and be activated by the physical entry of food into the stomach and/or by any of the means described herein. Alternatively, the sensor may be located outside the body and be activated by the patient by any of the means described herein before, during, or after the patient consumes food. Alternatively, the sensor may comprise a plurality of sensors in a neural network and may be activated by any means described herein.

As noted, the devices and processes according to the invention include a sensor that detects food in the stomach and communicates with and/or activates the GESD and/or the IESD. After the sensor communicates with and/or activates the electrical stimulation device or devices, the electrical stimulation device or devices artificially alters for preset periods of time, and preferably by means of sequential electrical pulses, the natural gastric motility of a patient by electrical stimulation. More preferably, such sequential electrical pulses are generated by an electrical stimulator which is applied by laparoscopic means to a portion of, or adjacent to, the stomach or small intestines. Even more preferably, such sequential electrical pulses are first applied to a portion of, or adjacent to, the stomach, followed after a predetermined delay, to a portion of, or adjacent to, the small intestines. Preferred locations for GESD include along the lower or distal end of the lesser curvature of the stomach. Preferred locations for IESD include along the duodenum and the jejunum. Of course, other portions of the stomach or small intestines can be electrically stimulated using the method of this invention. The sensor may be placed within or adjacent to the stomach (including placement internal or external to the body). The sensor can be used to trigger the GESD and/or IESD before, during, and/or after the consumption of food.

As indicated above, a wide variety of sensors can be used in the present devices and methods. For example, a sensor contained in a capsule or pill could be ingested by the patient at the same time or before food is consumed. A sensor located on the outside of the body could activated by the patient at the appropriate time (e.g., a predetermined period before or after beginning to eat or at the time of eating) which would, in turn, trigger the GESD and/or IESD; with such a sensor, the patient could determine if and when the sensor would be activated. For example, the patient might be instructed to activate the sensor only if a high calorie substance (solid or liquid) was to be consumed but not to activate if a low calorie substance (e.g., water, diet cola, or the like) was to be consumed.

Sensors which measures parameters, including physical, chemical, and or electrical parameters, which change during, or are involved in, digestion could also be used. Such sensors could include, but are not limited to, strain gauges to indicate or measure mechanical movement, thermistor or thermopile sensors to indicate temperature changes associated in food digestion or intake, an accelerometer to indicate stomach motion, or a piezoelectric crystal to indicate acoustic vibration or noise associated with food digestion, ultrasound or rf components to facilitate measurement of stomach distension and contraction, impedance plethymography to indicate stomach tissue changes associated with stomach distension and contraction and/or an such means or indicators described herein.

For example, sensors which monitor changes in the volume or expansion of the stomach could be used. One such sensor could be an elastic band around a portion of the stomach (e.g., passing around the stomach in the area of the lesser and greater curvatures). As the stomach is extended due to consumption of food, such a band would expand and activate the sensor at a predetermined expansion value; the sensor would, in turn and as programmed, activate the appropriate electrostimulation device or devices. Deactivation of the appropriate electrostimulation device or devices could occur, as desired, based on a predetermined time after activation or by contraction of the stomach as determined by the elastic band contracting to its normal, non-stretched state.

Alternatively, multiple sensors could be located spaced apart on the exterior of the stomach (e.g., fundus or other portions of the stomach and generally attached to the serosa), wherein the sensors can determine the distance separating them. As the stomach expands (as food fills the stomach) or contracts (as foods empties from the stomach), the distance between the sensors will change (i.e., generally increasing as the stomach expands and generally decreasing as the stomach contracts). Such multiple sensors could, for example, include one sensor to generate an a signal (e.g., ultrasound, magnetic field, and the like) and the other sensor to detect the signal, thereby allowing the distance between the two sensors to be determined. For such ultrasound sensors, the distance would be determined by measure the time between the transmission and detection of the ultrasound signal. For such magnetic field sensors, the distance would be determined by the strength of the magnetic filed measured at the detecting sensor; thus, an increase in the distance between the two sensors increases would result in a decrease in the magnetic field at the detecting sensor. As the distance increases above a predetermined level, the sensor would, in turn and as programmed, activate the appropriate electrostimulation device or devices. Deactivation of the appropriate electrostimulation device or devices could occur, as desired, based on a predetermined time after activation or by contraction of the stomach as determined by the sensor or sensors.

Sensors for measuring pH within the stomach could also be used. Such a pH sensor could be used to activate one or more electrostimulation devices when the pH, for example, falls below a certain value indicating ongoing digestive processes within the stomach. Deactivation could occur, as desired, based on a predetermined time after activation or by an increase in the pH back towards neutral (i.e., as acid is absorbed by the food and the proton pumps are not reactivated by processes or signals within the digestive system). Alternatively, sensors effective for detecting increases or decreases, as appropriate, in digestive enzymes or other chemicals associated with digestion could be used in essentially the same manner.

Motion sensors mounted on the stomach can also be used in order to detect peristaltic movement associated with digestion. As the motion of the stomach, as measured by the sensor, increases above a predetermined value, the sensor could activate one or more electrostimulation devices. Deactivation could occur, as desired, based on a predetermined time after activation or by an decrease in stomach motion indicating a non-digestive state.

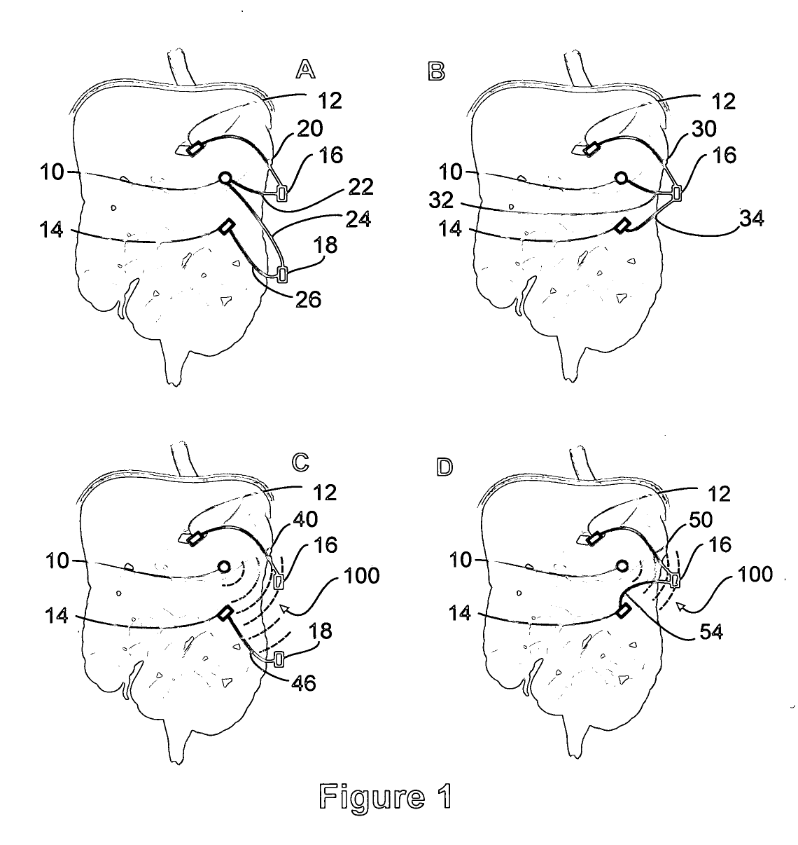

In order to further clarify the process and device for treating obesity and syndromes related to motor disorders of the stomach of a patient, according to the invention, FIGS. 1-4 are described. FIG. 1 includes sectional views of a gastrointestinal tract showing several non-limiting embodiments wherein the sensor 12, GESD 10, and/or the IESD 14 are located on various organs or locations within the gastrointestinal tract and/or wherein various communication pathways are shown. The organs in FIG. 1 are not specifically labeled as the identities of the various organs within the gastrointestinal tract are clear to one skilled in the art. In Panels A-D of FIG. 1, the sensor 12, which is located on the body of the stomach near the greater curvature, is activated by coordinated movement of the stomach indicating the beginning or continuation of the digestion process, activity associated with the gastric pacemaker, and/or by any of the means described herein. Of course, the sensor 12 could be located on other areas of the stomach or gastrointestinal tract so long as an appropriate activity related to the consumption of food or the digestive process can be detected. In Panel A, the activated sensor 12 communicates with a first pulse generator 16 through electrical pathway 22 and a second pulse generator 18 through electrical pathway 24. The first pulse generator 22 then communicates with GESD 12, located on, or adjacent to, the lesser curvature of the stomach via electrical pathway 20. The GESD 12 then supplies the appropriate electrostimulation of the stomach for a first predetermined period of time. The second pulse generator 13 communicates with IESD 14, which is located on, or adjacent to, the small intestine via electrical pathway 26. The IESD 14 supplies electrical stimulation to the patient's small intestine for a second predetermined period of time; the IESD can be placed on the small intestines in a position closer or further away from the stomach than shown in Panels A-D. Using this arrangement, electrical stimulation can be applied to the stomach and small intestines at the same time or in a sequential manner (e.g., first to the stomach for the first predetermined time and then to the small intestines after a predetermined delay time). The stimulation to the stomach and small intestines can be overlapping or non-overlapping in time.

Panel B of FIG. 1 is similar to Panel A except that the sensor 10 communicates, via electrical pathway 32, with a single first pulse generator 16 which, in turn, communicates with both the GESD 12 and the IESD 14 via electrical pathways 30 and 34, respectively. Operation would be similar to that described for Panel A above (i.e., electrical stimulation applied to the stomach and small intestines at the same time or in a sequential manner).

Panels D and C are similar to Panels A and B, respectively, except communication from the sensor 10 to the first and second pulse generators 16 and 18 in Panel C or the single pulse generator 16 in Panel D is carried out in a wireless manner (e.g., radio or other wireless signals 100). Except for the use of wireless communication, operation would be similar to that described for Panels A and B above (i.e., electrical stimulation applied to the stomach and small intestines at the same time or in a sequential manner).

Although not shown in FIG. 1, the sensor 10 can be located on other parts of or within the stomach, other locations within the gastrointestinal tract, as well as external to the body so long as an appropriate activity related to the consumption of food or the digestive process can be detected. For sensors placed within the stomach and/or external to the body, the use of wireless communication as illustrated in Panels C and D would, of course, be preferred. The sensor, which is in communication with the GESD and/or the IESD can also be used to automatically indicate, suggest, and/or terminate the resultant electrical stimulation and/or adjust or modify the parameters of the electrical stimulation. Although the embodiments shown in FIG. 1 include both electrostimulation of the stomach and the small intestines, the sensor could be used with only electrostimulation of the stomach or small intestines if desired.

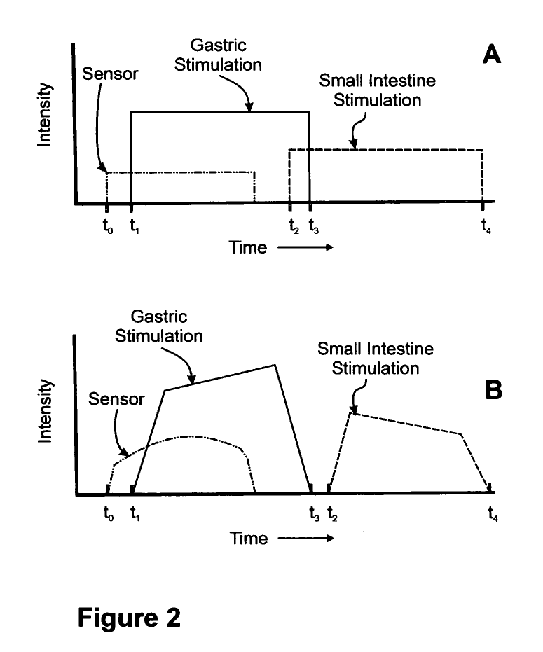

FIG. 2 illustrates several plots of the intensity of the response of the sensor detecting food intake as well as the electrostimulation impulses provided to the stomach and small intestines as a function of time in a preferred embodiment. In both Panels A and B of FIG. 2, the sensor detects entry of food into the stomach at time to, thereby triggering electrostimulation of the stomach at time t₁ and electrostimulation of the small intestines at time t₂. Electrostimulation of the stomach may begin immediately after detection of food entering the stomach (i.e., t₁-t₀=0) or after a delay (i.e., t₁-t₀>0). Although not shown in FIG. 2, the sensor may only activate the electrostimulators if activity associated with food ingestion continues for some preset time or if the total amount of food ingested (i.e., area under the sensor curve) reaches some preset amount; such modifications would reduce the instances of electrostimulation be triggered by transient events (e.g., limited food intake or non-food related events) recorded by the sensor. Electrostimulation of the stomach would continue until time t₃ (i.e., for a predetermined time t₃-t₁). Electrostimulation of the small intestines would begin at time t₂ (i.e., after a delay time of t₂-t₀) and continue for a predetermined time t₄-t₂). The gastric stimulation and small intestine stimulation may be overlapping or non-overlapping as shown in Panels A and B, respectively. Panel A shows both gastric stimulation and small intestine stimulation at constant (but different) intensities. Panel B shows gastric stimulation intensity as first increasing at a relatively fast rate, followed by increasing at a slower rate, and finally decreasing at a relatively fast rate and small intestine simulation and small intestine in as first increasing at a relatively fate rate, followed by decreasing at a relatively slow rate, and finally decreasing at a faster rate. Of course, other variables related to the electrostimulation (i.e., pulse width, frequency, and the like) could be varied in a similar manner. Moreover, the parameters of the electrostimulation could be varied on a daily, monthly, or even random basis in order to decrease the risk of the patient becoming conditioned to the electrostimulation. Indeed, periodically only one of the electrostimulation could be activated (i.e., either the stomach or the small intestines) to further decrease this risk.

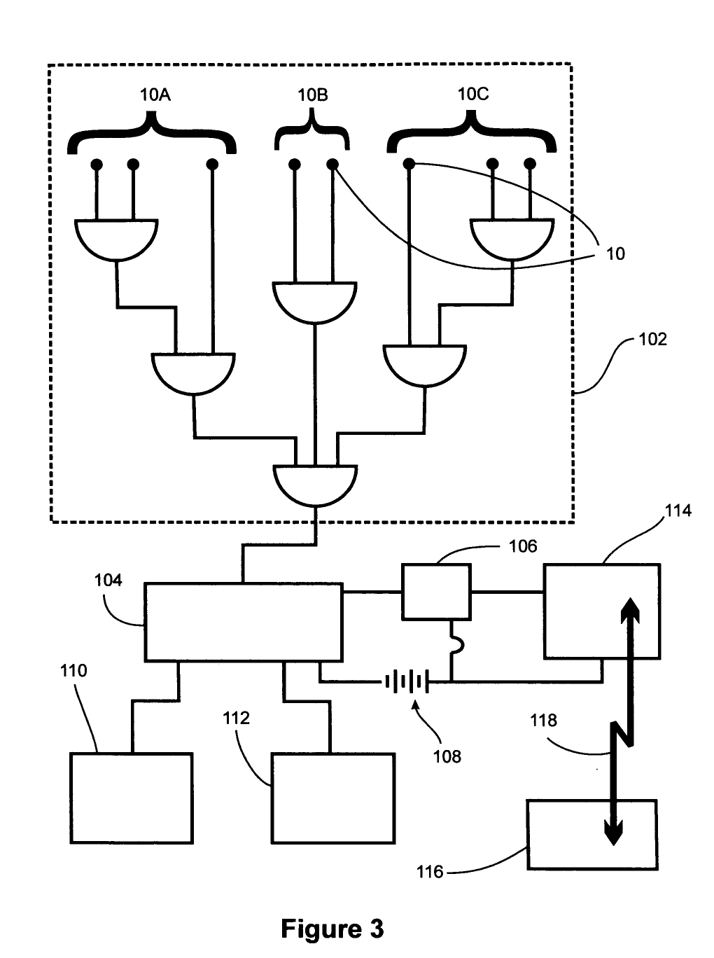

FIG. 3 is a block diagram showing a further embodiment of the invention using a plurality of sensors 10 in a sensor neural network 102 in communication with a microprocessor 104, with programmable random access memory 106, and powered by battery or a like power supply 108. Sensors 10A, 10B, and 10C can be, for example, sensors placed on or adjacent to the stomach, sensors placed within the body to record patient conditions, and sensors placed on or adjacent to the small intestines, respectively. Other sensor placement, different numbers of such sensors, and other types of activities being monitored can also be used. Preferably a rechargeable battery 108 is used; preferably such a battery 108 can be recharged without removing it from the body. The microprocessor 104 is in communication with at least one GESD 110 and/or at least one IESD 112. The microprocessor 104 serves as an information processor, information storage unit, and/or control unit which can be programed to provide automatic control and/or adjustments to the GESD 110 and/or IESD 112 and, thus, control or modify the applied therapy as desired. The microprocessor 104 is also in communication with a telemetry device 114 which, in turn, can be used to communication (e.g., provide a means to output data on the patient's condition and/or input data from the physician or other medical personnel to modify the therapy) with a processing device 116 located outside the body; such communication modes include, but are not limited to, two-way digital or radio communications. The processing device 116 telemeters instructions to and/or receives data from the telemeter device 114; preferably both the processing device 116 and telemeter device 114 can both transmit and receive signals from each other (as indicated by the bold line 1118). Accordingly, sensor neural network 102 provides for automatic control and synchronization of electrical stimulation parameters such as frequency, interval, amplitude, location, and/or any combination thereof related to the therapy. Data downloaded may be used to monitor the patient's condition and progress and, as appropriate, modify the parameters of the electrostimulation (see, e.g., FIG. 2A above). Data from such a network may be displayed in real time or stored for later analysis.

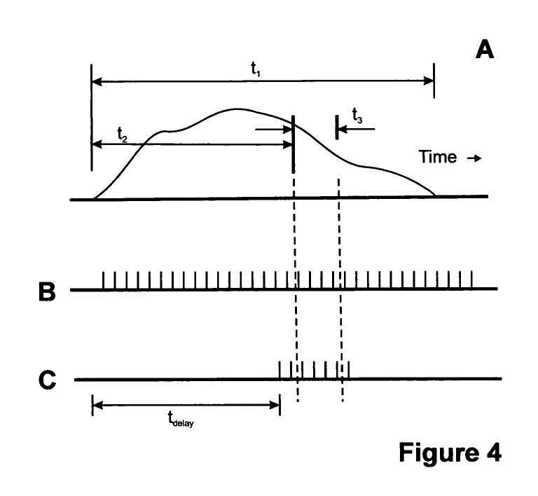

As noted above, gastrointestinal electrograms indicate an intrinsic gastric muscular electrical activity of the stomach as about 2 to about 4 slow waves per minute whereas the small intestine has an activity of about 10 to about 13 slow waves per minute. Generally spikes have a rate of about 120 to about 1200 waves per minute in both the stomach and the small intestine. The slow waves appear to act as a clock for contractile waves and determine the frequency and propagation direction of the contractile waves. FIG. 4A illustrates typical slow waves (duration t₁) and the window of susceptibility. The window of susceptibility occurs after a delay t₂ (i.e., from the onset of the slow wave to the window of susceptibility) and has a duration t₃. FIG. 4B provides a conventional, essentially continuous, pulse train used for electrostimulation (i.e., not synchronized with the window of susceptibility). Pulses applied during the window of susceptibility will be most effective (e.g., greatest probability of causing contractions in the stomach); pulses outside this window of susceptibility are wasted and may even be counterproductive (i.e., interfere with the efficiency of the intrinsic GI activity by depolarizing the tissue before an intrinsic contraction is initiated). Thus, the conventional stimulation approach illustrated in Panel B may result in the expenditure of needless energy and/or actually adversely affect the overall process. FIG. 4C illustrates stimulation using one embodiment of the present invention. In this embodiment, the sensor detects the slow wave (typical duration t, of about 20 to about 30 seconds) and initiates stimulation only after a time t_{delay} (typically about 4 to about 9 seconds) so that the stimulation is synchronized with the window of susceptibility, thereby increasing the efficiency of the stimulation. As also shown in Panel C, the stimulation is preferably turned off shortly after the window of susceptibility has ended. The window of susceptibility may be identified for a particular patient and coupled with the synchronized electrical stimulation as indicated for efficacy during the identified window of susceptibility. Accordingly, automatic, continuous, or periodic adjustment of parameters for the electrical stimulation by utilizing the window of susceptibility to optimize longevity and effectiveness of the therapy can be effected. Thus, sensor can be utilized for initiation, termination, and/or adjustment of the therapy through manipulation of parameters including, but not limited to, frequency, amplitude, burst duration, burst interval, polarity parameters indicated at the stomach wall, and the like.

As noted above, the sensor or sensors, in addition to initiating electrostimulation, may measure the physiological parameters and other patient information and transmit this information via unidirectional or bidirectional communication to a remote unit for display. Sensors may also be used to automatically verify that the appropriate electrical stimulation occurred by sensing of the intrinsic gastrointestinal electrophysiological activity. Using such information, the physician or other health provider may modify, as appropriate, the therapy based on the patient's progress, the patient's conditioning and/or tolerance to the therapy, effectiveness of the therapy, and the like. Modifications can be made to the therapy to assist the physician or other health providers to determine the optimal therapy for the individual patient. The system can also be designed to automatically modify the therapy over time to experiment, within well prescribed limits, with the operational parameters; the results obtained could be used assist the physician or other health providers to determine the optimal therapy for the individual patient.

Such recorded patient information may be used in treating patients who are experiencing difficulties, treating patients who are resistant to physician follow-up since the results can be reviewed by the physician using remote or telephonic means), or for maintaining patient records for use at a later time. The means of retrieving such information include but are not limited to: transtelephonic means (i.e., telephone interface device), an internet network, or wireless connection. Such network or wireless connections may provide downloading of information through a computer interface via modem or wireless transmission to a physician or monitoring service. Stimulator devices may also be programmed through a telephone or computer interface, which may automatically provide adaptive functions based on the history of successful stimulations versus delivered stimulation. The display and storage of such information is advantageous because the physician is better informed about the optimal stimulation intervals previously used to achieve increased efficacy and increased device longevity.

The present invention also allows for electrical stimulation to be applied in a proportional manner in relation to the patient's activity (typically using an additional activity sensor). For example, the activity sensor can measure the level of patient activity, for example whether the patient is sleeping, exercising, or performing daily tasks. In a low level of patient activity, such as sleeping, the activity sensor may turn off the electrical stimulation. Also the activity sensor can turn off, or reduce the intensity or duration of, the electrical stimulation if a patient's activity sensor indicates the patient is in distress. The activity sensor can measure physiological conditions such as heart rate, blood pressure; temperature, and other similar conditions. The activity sensor may also measure chemical conditions such a blood sugar or other chemical indicators.

An algorithm can be included in the system of this invention whereby stimulation parameters can automatically be adjusted based on the patient's condition and/or response to previous stimulation profiles. The stimulation parameters can be automatically, periodically, or continuously adjusted within a physician's defined range. Thus, the system may select, suggest, telemeter, and/or adjust stimulation parameters to optimize longevity and efficacy of specific patient therapy.

The GESD and/or IESD devices used in the present invention preferably have preset operating frequencies and periods which may be varied according to the alteration of stomach motility to be obtained and/or to the pathological condition of the patient. Generally, the GESD has an operating frequency of about 2 to about 15 pulses per minute. Preferably, the GESD of this invention employs stimulation of the stomach at a rate of about 2 to about 15 pulses/minute with each pulse lasting about 0.5 to about 4 seconds such that there is a pause of about 3 to about 30 between the pulses. More preferably, the pulse rate is about 12 pulses/minute with each pulse lasting about 2 seconds with a pause of about 3 seconds between pulses. Preferably, the pulse amplitude is about 0.5 to about 15 milliamps. More preferable, each pulse consists of a train of micro-bursts with a frequency of about 5 to about 100 sect⁻¹. Preferably, the IESD of this invention employs stimulation of the small intestines at a rate of about 2 to about 15 pulses/minute with each pulse lasting about 0.5 to about 4 seconds such that there is a pause of about 3 to about 30 seconds between the pulses. The electrical discharge of each pulse can vary from approximately about 1 to about 15 volts for voltage-controlled stimulation and from about 2 to about 15 milliamperes for constant current stimulation. More preferably, the pulse rate of the IESD is about 12 pulses/minute with each pulse lasting about 2 seconds with a pause of about 3 seconds between pulses. Preferably, the pulse amplitude is about 0.5 to about 15 milliamps. More preferable, each pulse consists of a train of micro-bursts with a frequency of about 5 to about 100 sec⁻¹. Other parameters can be used and may, for particular patients, be preferred.

Generally conventional laparoscopic or minimally invasive surgical techniques are used to place the various components in the appropriate locations within the body and, preferably, within the abdominal cavity. Conventional electrical stimulation devices may be used in the practice of this invention. Such devices include, for example, those described in U.S. Pat. No. 5,423,872 (Jun. 3, 1995); U.S. Pat. No. 5,690,691 (Nov. 25, 1997); U.S. Pat. No. 5,836,994 (Nov. 17, 1998); U.S. Pat. No. 5,861,014 (Jan. 19, 1999); PCT Application Serial No. PCT/US98/10402 (filed May 21, 1998) and U.S. patent application Ser. No. 09/424,324 (filed Jan. 26, 2000); U.S. Pat. No. 6,041,258 (Mar. 21, 2000); U.S. patent application Ser. No. 09/640,201 (filed Aug. 16, 2000); PCT Application Serial No. PCT/US00/09910 (filed Apr. 14, 2000) entitled “Gastric Stimulator Apparatus and Method for Installing” based on U.S. Provisional Application Ser. Nos. 60/129,198 and 60/129,199 (both filed Apr. 14, 1999); PCT Application Serial No. PCT/US00/10154 (filed Apr. 14, 2000) entitled “Gastric Stimulator Apparatus and Method for Use” based on U.S. Provisional Application Ser. Nos. 60/129,209 (filed Apr. 14, 1999) and 60/466,387 (filed Dec. 17, 1999); and U.S. Provisional Patent Application Ser. No. 60/235,660 (filed Sep. 26, 2000) entitled “Method and Apparatus for Intentional Impairment of Gastric Motility and/or Efficiency by Triggered Electrical Stimulation of the Gastric Tract with Respect to the Intrinsic Gastric Electrical Activity.” All of these patents, patent applications, provisional patent applications, and/or publications as well as any mentioned elsewhere in this specification are hereby incorporated by reference.

In addition to initiating or activating the electrostimulation, a sensor (either the same sensor used to activate the electrostimulation or a separate sensor) could also be used, in appropriate cases, be used to signal the patient in order to help modify behavior. Where a patient, for example, continues to eat in spite of the electrostimulation and the resulting feeling of satiety, a sensor to monitor the distention of the stomach (or other suitable parameters) could provide a detectable signal (e.g., vibration, sound, and the like) to the patient (via, for example, a receiving device the patient carries) when the distension reaches a predetermined level (higher than that which activate electrostimulation); such a signal would positive feedback to the patient that he or she should stop eating and, working in combination with the electrostimulation, assist the patient to modify the undesirable behavior (i.e., ignoring the feeling of satiety). Alternatively, the sensor could measure the rate of distention and, where appropriate (i.e., when the rate exceeds some predetermined value), provide a signal to indicate the patient should eat slower. The predetermined levels in such methods could be lower over time to assist the patient to adopt more reasonable eating habits.

Although other types may be used, preferred electrical stimulation devices include electrocatheters having an elongated body with a distal end having an electrical stimulation lead or leads mounted on, or attached to, the stomach in the region of the lesser curvature and a proximal end for attachment to a pulse generator. The electrical stimulation lead or leads are attached to a power source through, or with, the pulse generator. Such preferred electrical stimulation devices are described in, for example, PCT Application Serial Number PCT/US98/10402 (filed May 21, 1998), U.S. patent application Ser. No. 09/424,324 (filed Jan. 26, 2000), and U.S. patent application Ser. No. 09/640,201 (filed Aug. 16, 2000). Of course, care should be taken in placement or attachment of the electrical stimulation device to avoid physical strangulation or other damage to the tissue of the intestines (especially with the region of the small intestine).

The present methods using a sensor or sensors to detect food entering the stomach or the initiation of digestive processes within the stomach can also be used with the methods and devices disclosed in our co-pending Provisional Application Ser. No. 60/557,737, filed on the same date as the present application, and entitled “Tachygastrial Electrical Stimulation” (Docket 74185), which is incorporated by reference in its entirety.

The methods, processes, sensors, and electrical stimulators used are susceptible to numerous modifications and variations, all of which are within the scope of the present inventive concept. Furthermore, all the details may be replaced with technically equivalent elements. The materials employed, the shapes, and the dimensions of the specific electrical stimulators may be varied according to the requirements.

The following examples are provided to describe the invention and not to limit it.

### EXAMPLE 1

### In a system comprising a sensor, a microprocessor, and one or more GESDs and/or IESDs, the following method of operation may be used:

read lead/tissue impedance;
read baseline gastric activity and patient condition;
   direct electrical input;
   non-electrode sensor input;
   patient condition sensor input (time since last meal, activity);
   adjust amplifiers to minimum level (e.g., ⅓ scale);
set initial stimulation parameters (high levels from look up table, constant stimulation string);
deliver stimulation string;
check sensor inputs for peristaltic response (e.g., greater than half scale, if saturated adjust amps);
sample sensor for minimum period of time (e.g., 10 seconds) allowing contraction to propagate to the sensor location;
   no response, telemeter to physician to adjust;
   response, decrease parameters and repeat stimulation and check sensor inputs;
repeat until peristaltic response is lost;
increase parameters two steps per look up table;
telemeter suggested parameters to physician or set automatically depending on mode setting;
   adjust stimulation synchronization to optimize slow wave window response;
   read sensor null (i.e., zero crossings);
   iterate stimulation delay from null until peristaltic; response occurs dependably/repeatedly; and
set maximum adaptive adjustment rate desired (device may repeat test in increments programmed. If response is valid, device will increase time to next test. If response is negative, device will automatically adjust parameters and retest at existing rate/interval).
automatically adjust parameters and retest at existing rate/interval).

### EXAMPLE 2

Information obtained from the sensor can be used to determine the optimal period for therapy or the window of susceptibility. The following description provides one method to accomplish this:
set stimulation parameters;
allow device to determine a baseline zero-crossing level (i.e., this should correspond to the interval of the slow waves);
device sets an initial stimulation interval/delay (minimum) from the zero-crossing;
device senses the next zero-crossing and delivers an abbreviated electrical pulse train after the initial delay;
device senses if a peristaltic contraction occurs;
   no peristaltic contraction, increase delay interval and repeat until contraction is achieved or stimulation interval corresponds to the length of a slow wave minus the minimum delay or until a second zero-crossing is sensed;
   peristaltic contraction, set delay; and
periodically check stimulation synchronization and adjust, if necessary (programmable as adaptive adjustment rate).

1. A method for treatment of a motor disorder in a patient, said method comprising:
(1) implanting a first electrical stimulation device comprising one or more first electrical stimulation leads and a first electrical connector for attachment to a first pulse generator such that the one or more first electrical stimulation leads are attached to, or adjacent to, the patient's stomach, whereby electrical stimulation can be provided to the stomach through the one or more first electrical stimulation leads;
(2) implanting a second electrical stimulation device comprising one or more second electrical stimulation leads and a second electrical connector for attachment to a second pulse generator such that the one or more second electrical stimulation leads are attached to, or adjacent to, the patient's small intestines, whereby electrical stimulation can be provided to the patient's small intestines through the one or more second electrical stimulation leads;
(3) placing a sensor in or near the patient's stomach, wherein the sensor can be activated when food enters the patient's stomach, wherein the sensor, when activated, can communicate with the first pulse generator and cause the first pulse generator to supply electrical stimulation to the patient's stomach for a first predetermined period of time and wherein the sensor, when activated, can communicate with the second pulse generator and cause the second pulse generator to supply electrical stimulation to the patient's small intestines, after a predetermined delay period, for a second predetermined period of time;
(4) supplying electrical stimulation to the patient's stomach through the one or more first electrical stimulation leads for the first predetermined period of time when the sensor is activated by a means inside or outside the patient's body; and
(5) supplying electrical stimulation to the patient's small intestines through the one or more second electrical stimulation leads after the predetermined delay period and for the second predetermined period of time sensor when the sensor is activated by a means inside or outside the patient's body.

2. The method of claim 1, wherein the sensor is attached to or adjacent to the outer stomach wall.

3. The method of claim 1, wherein the sensor is ingested in an oral capsule/pill form before, during, or after food is consumed.

4. The method of claim 1, wherein the sensor is located outside the body.

5. The method of claim 1, wherein the sensor comprises a plurality of sensors in a neural network.

6. The method of claim 1, wherein the sensor may communicate physiological parameters or patient information to a remote unit.

7. The method of claim 1, wherein the sensor automatically selects stimulation parameters according to an incorporated algorithm.

8. The method of claim 1, wherein the sensor adjusts the stimulation parameters or suggests adjustments to the stimulation parameters, whereby a health care provider and/or the patient may adjust the stimulation parameters.

9. The method of claim 1, wherein the electrical stimulation applied to the patient is applied in a proportional percentage in relation to the sensor information.

10. The method of claim 1, further comprising an activity sensor, wherein the electrical stimulation applied to the patient is applied in a proportional percentage in relation to the activity sensor information.

11. A method for treatment of a motor disorder in a patient, said method comprising
(1) implanting an electrical stimulation device comprising one or more electrical stimulation leads and an electrical connector for attachment to a pulse generator such that the one or more electrical stimulation leads are attached to, or adjacent to, the patient's stomach, whereby electrical stimulation can be provided to the stomach through the one or more electrical stimulation leads;
(2) placing a sensor in or near the patient's stomach, wherein the sensor can be activated when food enters the patient's stomach, wherein the sensor, when activated, can communicate with the pulse generator and cause the pulse generator to supply electrical stimulation to the patient's stomach for a predetermined period of time; and
(3) supplying electrical stimulation to the patient's stomach through the one or more first electrical stimulation leads for the predetermined period of time when the sensor is activated by a means inside or outside the patient's body.

12. The method of claim 11, wherein the sensor is attached to or adjacent to the outer stomach wall.

13. The method of claim 11, wherein the sensor is ingested in an oral capsule/pill form before, during, or after food is consumed.

14. The method of claim 11, wherein the sensor is located outside the body.

15. The method of claim 11, wherein the sensor comprises a plurality of sensors in a neural network.

16. The method of claim 11, wherein the sensor may communicate physiological parameters or patient information to a remote unit.

17. The method of claim 11, wherein the sensor automatically selects stimulation parameters according to an incorporated algorithm.

18. The method of claim 11, wherein the sensor adjusts the stimulation parameters or suggests adjustments to stimulation parameters, whereby a physician and/or patient may adjust the stimulation parameters.

19. A method for treatment of a motor disorder in a patient, said method comprising
(1) implanting an electrical stimulation device comprising one or more electrical stimulation leads and a electrical connector for attachment to a pulse generator such that the one or more electrical stimulation leads are attached to, or adjacent to, the patient's small intestines, whereby electrical stimulation can be provided to the patient's small intestines through the one or more electrical stimulation leads;
(2) placing a sensor in or near the patient's stomach, wherein the sensor can be activated when food enters the patient's stomach, wherein the sensor, when activated, can communicate with the pulse generator and cause the pulse generator to supply electrical stimulation to the patient's small intestines, after a predetermined delay period, for a predetermined period of time; and
(3) supplying electrical stimulation to the patient's small intestines through the one or more electrical stimulation leads after the predetermined delay period and for the predetermined period of time sensor when the sensor is activated by a means inside or outside the patient's body.

20. The method of claim 19, wherein the sensor is attached to or adjacent to the outer stomach wall.

21. The method of claim 19, wherein the sensor is ingested in an oral capsule/pill form before, during, or after food is consumed.

22. The method of claim 19, wherein the sensor is located outside the body.

23. The method of claim 19, wherein the sensor comprises a plurality of sensors in a neural network.

24. The method of claim 19, wherein the sensor may communicate physiological parameters or patient information to a remote unit.

25. The method of claim 19, wherein the sensor automatically selects stimulation parameters according to an incorporated algorithm.

26. The method of claim 19, wherein the sensor adjusts the stimulation parameters or, suggests adjustments to stimulation parameters, whereby a physician and/or patient may adjust the stimulation parameters.

27. The method of claim 19, wherein the sensor comprises a plurality of sensors in a neural network.

28. Method for treatment of a motor disorder in a patient, said method comprising:
(1) implanting an electrical stimulation device comprising an information processor, a plurality of sensors, and an electrical stimulator;
(2) placing the information processor in communication with the plurality of sensors, wherein the plurality of sensors provide physiological parameter(s) and/or patient condition information to the information processor; and
(3) placing the information processor in communication with the electrical stimulator, wherein the information processor operates the electrical stimulator and wherein the electrical stimulator can provide electrical stimulation to the patient's stomach and/or small intestines through the one or more electrical stimulation leads.

29. Method for treatment of a motor disorder in a patient, said method comprising:
(1) implanting an electrical stimulation device comprising an telemeter, a plurality of sensors, and an electrical stimulator;
(2) placing the telemeter in communication with the plurality of sensors, wherein the plurality of sensors provide physiological parameter information or patient condition information to the telemeter; and
(3) placing the telemeter in communication with the electrical stimulator, wherein the telemeter operates the electrical stimulator and wherein the electrical stimulator can provide electrical stimulation to the patient's stomach and/or small intestines through the one or more electrical stimulation leads.

30. Method for treatment of a motor disorder in a patient, said method comprising:
(1) implanting an electrical stimulation device comprising an information processor, a plurality of sensors, a telemeter, and an electrical stimulator;
(2) placing the information processor in communication with the plurality of sensors, wherein the plurality of sensors provide physiological parameter(s) and/or patient condition information to the information processor;
(3) placing the information processor in communication with the telemeter; and
(4) placing the telemeter in communication with the electrical stimulator, wherein the telemeter operates the electrical stimulator and wherein the electrical stimulator can provide electrical stimulation to the patient's stomach and/or small intestines through the one or more electrical stimulation leads.

31. Method for treatment of a motor disorder in a patient, said method comprising:
(1) implanting an electrical stimulation device comprising an information processor, a sensor, and an electrical stimulator;
(2) placing the information processor in communication with the sensor, wherein the sensor detects slow waves associated with a visceral gastrointestinal organ and provides data regarding the slow waves of the visceral gastrointestinal organ and its window of susceptibility to the information processor; and
(3) placing the information processor in communication with the electrical stimulator, wherein the information processor, using the data regarding the slow waves provided by the sensor, operates the electrical stimulator to provide electrical stimulation to the gastrointestinal organ which is synchronized with the window of susceptibility associated with the slow waves,
wherein the gastrointestinal organ is the patient's stomach or small intestines.

32. The method of claim 31, wherein the electrical stimulation device automatically, continuously, or periodically adjusts the electrical stimulation utilizing the window of susceptibility to improve longevity of the electrical stimulation device or effectiveness of the method.
